(19) United States
(12) Patent Application Publication
(10) Pub. No.: US 2013/0302298 A1
(43) Pub. Date: Nov. 14, 2013

(54) TREATMENT AND PREVENTION OF ECZEMA

(71) Applicants: Claude Chevalier, Quebec (CA); Serge Carriere, Quebec (CA)

(72) Inventors: Claude Chevalier, Quebec (CA); Serge Carriere, Quebec (CA)

(73) Assignee: BIO-K PLUS INTERNATIONAL INC., Quebec (CA)

(21) Appl. No.: 13/990,358

(22) PCT Filed: Nov. 29, 2011
(86) PCT No.: PCT/CA2011/001319

§ 371 Date: Jul. 26, 2013

Related U.S. Application Data
(60) Provisional application No. 61/418,158, filed on Nov. 30, 2010.

(51) Int. Cl. A61K 35/74

(52) U.S. Cl. CPC A61K 35/747
USPC 424/93.45

(57) ABSTRACT

The present invention broadly relates to the use of probiotics for the treatment and prevention of dermatitis. More particularly, the present invention relates to the use of a composition comprising Lactobacillus acidophilus strain I-1492 deposited at the CNCM, a Lactobacillus casei LBC80R® strain and a carrier for treating or preventing hand dermatitis.

### TECHNICAL FIELD

The present invention broadly relates to the use of probiotics for the treatment and prevention of dermatitis. More particularly, the present invention relates to the use of a composition comprising Lactobacillus acidophilus strain I-1492 deposited at the CNCM, a Lactobacillus casei strain and a carrier for treating or preventing hand dermatitis.

### BACKGROUND OF THE INVENTION

Hand eczema, also known as hand dermatitis (HD), is a relatively common inflammatory skin disease affecting roughly 10% of the population and is characterized by dryness, erythema, scaling, pruritis and pain, that is often induced by contact with allergens or irritants.

HD can be extremely debilitating both physically and emotionally negatively impacting the patient's quality of life. As such, HD is a significant social burden for patients. Among other things, many patients feel stigmatized because of the visibility of the disease. Patients feel that others lack understanding and almost 50% of hand eczema sufferers have suspected other people believe the disease was contagious. It is also known that patients who suffer from chronic HD have low self-esteem, increased anxiety and social phobia. 80% of patients report that hand dermatitis affects their daily life, with women generally more affected than men. 72% report that they feel handicapped from leisure activities. 56% state that they have changed their daily activities as a result of their hand dermatitis. 36% report sleep disturbance with 46% reporting mood disturbance. Patients with moderate to severe hand dermatitis report significant negative impact, with a relationship between the severity of HD and the quality of life.

HD also represents a significant economic burden. With respect to the socio-economic impact of occupational hand eczema is the most frequently recognized occupational disease. It is expensive with respect to Worker Compensation and patients with HD are more likely to report long term sick leave (12%), job loss (8.5%), and lower quality of life. Patients also have lower socio-economic status because of the increased sick leave and job loss. In severe cases of HD the quality of life is comparable to that of atopic eczema and psoriasis. The disease burden is compounded in sufferers of occupational HD by a need to alter their work environments, which is often insufficient resulting in 8% changing their occupation and 15% voluntarily or involuntarily losing their jobs, highlighting the negative financial impact that HD can also have.

The causes of eczema (atopic dermatitis) are not yet fully known and are believed to be “multifactorial”. Hand eczema often results from a combination of genetic makeup (genetic or constitutional factors), environmental components (e.g. contact with irritants) and allergies. The genetic component of the disease is believed to arise from interplay of multiple genes with the external environmental factors. The more atopic genes that are present, the less environmental initiators are required to produce eczema. Further the genetic defect in the epidermal barrier is present, makes the skin susceptible to breakdown by irritants, such as detergents and dust mites, which may allow increased penetration of antigens. Further, a higher incidence of HD has been associated with certain occupations (occupational dermatitis), such as those involving frequent ‘wet work’ or contact with irritants and allergens (i.e., hairdressers, nurses, etc).

Studies have shown that the serum levels of IgE, immunoglobulins that are produced in response to antigens or allergens, are elevated in patients with eczema. Several genes have been associated with increased production of IgE and IgE can be measured and used as a marker of allergic type conditions. IgE levels are often related to the severity of eczema. However, 20% of atopic eczema have normal or low levels of IgE. There is a reduction in the cell-mediated immune response; chemotaxis of neutrophils and monocytes is reduced. These two last effects would explain the increased risk of infection of the skin in this condition.

Studies also suggest that there is excessive stimulation of T cells in eczema, which in turn increases the production of IL-4 (interleukin 4). Elevation of IL-4 and IL-13 may inhibit the antimicrobial—peptide gene expression which is part of the protective shield allowing proliferation of Staphylococcus aureus on and within the surface keratinocytes. There is also an increase in Langerhans cells in the epidermis. These usually present antigens to the immune system.

Further, macrophages in this condition increase the production of IL-10 which, in turn, stimulates TH2 cytokine response. The fundamental cause is still elusive. There is a relative immune deficiency in the skin producing an increased risk of secondary infection. Also, a reduction in the barrier function of the epidermis, which is likely genetic, would increase water loss and subsequent dryness of the skin.

Mild forms of HD are generally well-controlled by irritant/allergen avoidance, skin moisturizers, keratolytic agents, emollients and low potency corticosteroids, but these treatments tend to be ineffective for more severe HD. In these more severe cases, topical treatments may consist of higher potency corticosteroids, retinoids or calcineurin inhibitors. Phototherapy, systemic corticosteroids and retinoids (alitretinoin) may also be required in some cases. However, some patients remain refractory to the above treatments and the side effects associated with their long-term use tend to limits their utilization to acute flare management, suggesting a need for alternative treatment strategies for effective disease control.

In recent years, studies have evaluated the potential benefits of ‘protective’ or probiotic microorganisms (i.e. bacteria and yeast) in promoting overall health and preventing or ameliorating diseases. These studies were stimulated by the theory that in certain diseases, the balance of intestinal microbiota favored development or proliferation of ‘harmful’ intestinal bacteria species. For instance, elevated numbers of harmful Bacteroides, Escherichia coli, Fusobacterium and enterococci, and correlative reduced levels of beneficial Bifidobacterium and Lactobacillus species, were found in the mucosa of patients with inflammatory bowel disease. Further, reduced levels of Bifidobacterium species are observed in feces of infants with eczema. Still according to this theory, the ingestion of probiotics would restored the microbial balance or favor the growth of ‘protective’ bacterial species. For instance, probiotic formulations proved effective in the treatment of mild pouchitis and in promoting remission of mild to moderate ulcerative colitis.

Although the exact mechanism of action of probiotics is currently unknown, there is some evidence that probiotic species would positively influence both local and systemic inflammation. This lead to multiple studies relating to the use of probiotics in various inflammatory conditions, including the evaluation of the potential benefits of probiotics for the treatment of atopic dermatitis.

While the use of probiotics may be a promising avenue in the treatment or prevention of HD, clinicians and patients face practical difficulties in using them. Indeed, the various studies have shown that the clinical effects of probiotics in treatment or prevention of diseases depend largely on the specific probiotic bacteria. To add further complexity to their use in the treatment of HD, clinical effects of probiotics have been shown to be specific to particular strains. In other words, even within same genus and species, it is difficult, or even impossible to predict the clinical effects of the various strains. Therefore, conclusion as to the beneficial effects of specific probiotic genus, species and strains cannot be extended to every probiotics.

### BRIEF SUMMARY OF THE INVENTION

According to one embodiment, there is provided a method of treating or preventing dermatitis in an animal. In this embodiment, the method comprises administering to said animal a composition comprising at least one Lactobacillus acidophilus strain and a Lactobacillus casei strain.

According to another embodiment, there is provided a composition for use in treating or preventing dermatitis in an animal, wherein the composition comprises at least one Lactobacillus acidophilus strain and a Lactobacillus casei strain.

According to a further embodiment, there is provided the use of a composition comprising at least one Lactobacillus acidophilus strain and a Lactobacillus casei strain in the manufacture of a medicament for treating or preventing dermatitis in an animal.

In one aspect, the at least one Lactobacillus acidophilus strain comprises the Lactobacillus acidophilus strain I-1492 deposited at the CNCM.

In another aspect, said at least one Lactobacillus acidophilus strain comprises the Lactobacillus acidophilus strain I-1492 deposited at the CNCM and a Lactobacillus acidophilus strain other than the Lactobacillus acidophilus strain I-1492 deposited at the CNCM.

In a further aspect, said Lactobacillus casei strain comprises Lactobacillus casei LBC80R® strain.

In yet another aspect, the composition further comprises a biologically acceptable carrier. Preferably, said biologically acceptable carrier is selected from the group consisting of a pharmaceutically acceptable carrier and a nutritionally acceptable carrier.

In still another aspect, said animal is a mammal, wherein the mammal is preferably a human.

In a further aspect, said composition is suitable for oral consumption. In this aspect, the composition is preferably a food product. The food product may be selected from the group consisting of a lyophilized powder, a dairy-based product, a soy-based product and a rice-based product, wherein the dairy-based product is preferably a lactic ferment product. In one aspect, the composition is preferably Bio-K+® capsules.

According to yet a further aspect, the composition comprises at least 10 billions colony forming unit (CFU) of bacteria, preferably at least 12.5 billions colony forming unit (CFU) of bacteria, more preferably at least 25 billions colony forming unit (CFU) of bacteria, even more preferably at least 30 billions colony forming unit (CFU) of bacteria, still even more preferably, at least 50 billions colony forming unit (CFU) of bacteria, and most preferably at least 100 billions colony forming unit (CFU) of bacteria.

According to a different aspect, said composition is administered in a single dose.

According to another aspect, said dermatitis is eczema, and preferably hand eczema. According to this aspect, said eczema is a mild eczema, a moderate eczema or a severe eczema.

According to another aspect, the invention comprises a kit for prevention or treatment of dermatitis wherein the kit comprises at least one container containing the composition of the invention. Preferably, the kit of the invention further comprising at least one element selected from the group consisting of a cleanser and a moisturizer.

### BRIEF DESCRIPTION OF THE FIGURES

In order that the invention may be readily understood, embodiments of the invention are illustrated by way of example in the accompanying drawings.

FIG. 1 is a schematic illustration of the evolution of the cohort of patients during the studies.

Further details of the invention and its advantages will be apparent from the detailed description included below.

### DETAILED DESCRIPTION

In the following description of the embodiments, references to the accompanying drawings or examples are by way of illustration of examples by which the invention may be practiced. It will be understood that other embodiments may be made without departing from the scope of the invention disclosed.

According to one embodiment, there is provided a method of treating or preventing dermatitis in an animal, and preferably a mammal, i.e. any living organism, which can suffer from dermatitis, including vertebrate such as in particular notably, human beings, domestic and wild animals.

In this embodiment, the method comprises administering a probiotic to the animal. According to the World Health Organization, probiotics are live micro-organisms which when administered in adequate amounts confer a health benefit on the host (Report of a joint FAO/WHO Working Group. Guidelines for the Evaluation of Probiotics in Food. London, Ontario, Canada: FAO/WHO; 2002). Probiotics are known to help intestinal bacteria perform their tasks more efficiently. They are also known to take over when the intestinal bacteria are weakened or even destroyed by antibiotics, stress, poor nutrition or any other factors. Accordingly, the term “probiotoc” is intended to broadly mean live microorganisms, including Lactobacillus species, Bifidobacterium species and yeasts, that may beneficially affect the host upon ingestion by improving the balance of the intestinal microflora.

The term “dermatitis” as intended herein means any inflammation of the skin and includes without limitation any rash, including psoriasis, seborrhea, atopic dermatitis (eczema) and the like. A person skilled in the art will appreciate that the causes of eczema, hand dermatitis and other types of dermatitis are generally the same and that treatment proven effective for one type of dermatitis (e.g. hand eczema) are most of the time also effective in treating other types of dermatitis. A person skilled in the art will appreciate that dermatitis can occur on any part of an animal or human body, including the hands (hand eczema or HD). A person skilled in the art will appreciate that atopic dermatitis or eczema is a particular type of inflammatory reaction of the skin causing itching and burning. The person skilled in the art will also appreciate that the symptoms of eczema are typically vesicles (i.e. small blister-like raised areas) in the first stage followed by erythema (reddening), edema (swelling), papules (bumps), and crusting of the skin followed by lichenification (thickening) and scaling of the skin.

The person skilled in the art will appreciate that eczema can vary from a mild eczema, a moderate eczema or a severe eczema. Accordingly, the term eczema as intended herein extends to any eczema, regardless of its severity. In one embodiment, the method and the composition are suitable for treating or preventing hand eczema in a mammal, regardless of the condition, i.e. whether the eczema is mild, moderate or severe.

The terms “prevent”, “prevention” and similar terms are intended to broadly mean a process by which dermatitis is eradicated or slowed.

By “treat”, “treating”, “treatment” or similar terms, the inventors intend to mean a process by which dermatitis or the symptoms thereof are maintained at a constant level, reduced or completely eliminated. As used herein, “treatment” means any manner in which the symptoms of conditions, disorder or disease are ameliorated or otherwise beneficially altered. Treatment also encompasses any pharmaceutical use of the compositions herein. “Treatment” also refers to both therapeutic treatment and prophylactic or preventative measures. Those in need of treatment include those already with the disorder as well as those prone to have the disorder or those in which the disorder is to be prevented. As used herein, the term “treating a bacterial infection” refers to a process whereby the metabolic activity of a bacterium or bacterial population in a host, preferably a mammal, more preferably a human, is inhibited or ablated.

According to one embodiment, the composition for use in treating or preventing dermatitis comprises at least one Lactobacillus acidophilus strain and a Lactobacillus casei strain.

In one embodiment, the at least one Lactobacillus acidophilus strain comprises the Lactobacillus acidophilus strain I-1492 deposited at the CNCM. The Lactobacillus acidophilus strain I-1492 can be also designated as the Bio-K+ Lactobacillus acidophilus CL1285® strain. Accordingly, the person skilled in the art will appreciate that for the purpose of this specification, the terms “Lactobacillus acidophilus strain I-1492” and “Lactobacillus acidophilus CL1285®” or similar terms can be used interchangeably or simultaneously to designate the same Lactobacillus acidophilus strain. In an alternate embodiment, the at least one Lactobacillus acidophilus strain comprises the Lactobacillus acidophilus strain I-1492 deposited at the CNCM and a Lactobacillus acidophilus strain other than the Lactobacillus acidophilus strain I-1492 deposited at the CNCM. In a further embodiment, the Lactobacillus casei strain comprises Lactobacillus casei LBC80R® strain.

In one embodiment, the composition may further comprise a biologically acceptable carrier. The terms “acceptable vehicle” or “acceptable carrier” refer to any non-medical ingredients in the composition and may be used for different functions. For example, it can be used as a preservation, solubilizing, stabilizing, emulsifying, softening, coloring, odoring, or as an antioxidizing agent. Preferably, the biologically acceptable carrier is selected from the group consisting of a pharmaceutically acceptable carrier and a nutritionally acceptable carrier. Examples of possible excipients or carriers are cellulose, ascorbic acid, magnesium stearate, silicon dioxide and titanium dioxide.

By “pharmaceutically acceptable”, the inventors intended to broadly mean a vehicle, which may be administered without any risk to a mammal, in particular to a human being, and this with few or no negative or toxic secondary effects. Such a vehicle may be used for different functions. For example, it can be used as a preservation, solubilizing, stabilizing, emulsifying, softening, coloring, odoring, or as an antioxidizing agent. These types of vehicles may be easily prepared by using methods well known to a person skilled in the art.

The term “nutritionally acceptable”, is intended to broadly mean a vehicle that can be administered without risk to a mammal, in particular to a human, and this with little or no negative or toxic side effects. Such a vehicle can be used for different functions. For example, it can be used as preservation, solubilizing, stabilizing, emulsifying, softening, coloring, odoring agent, or as an antioxidant agent. These types of vehicles may be easily prepared by using methods well known by a person in the art.

In a preferred embodiment the composition of the invention comprises at least about 0.5 billion of living and active micro-organisms of the L. acidophilus strain per gram of the composition, up to about 143 days under refrigeration. In a more preferred embodiment the composition of the invention comprises about 50 billions, of a population of living and active micro-organisms of the L. acidophilus strain, per unit of the composition, up to about 143 days under refrigeration. The composition of the invention may also comprise at least about 100 billions of a population of living and active micro-organisms of the L. acidophilus strain, per unit of the composition, up to about 90 days under refrigeration, By “unit”, it is broadly meant any container suitable for commercial use, which contains about 98 grams of the composition of the invention, such as, but not limited to, a jar or a plastic container usually used for containing dairy products such as yogurts, or other ferments.

According to another preferred embodiment of the invention, the composition comprises the Bio-K+® products. According to yet another preferred embodiment of the invention, the lactic composition of the invention further comprises fermented milk proteins and fermented soy proteins. Bio-K+® products are lactic ferment products readily available on the market and sold by the company Bio-K Plus International Inc. The Bio-K+® products contain Lactobacillus acidophilus and Lactobacillus casei, and more specifically the Lactobacillus acidophilus strain I-1492 deposited at the CNCM and Lactobacillus casei LBC80R® strain.

A person skilled in the art will know how to prepare compositions that are nutritionally acceptable and determine, in function of many factors, the privileged method of administration and the quantity that should be administered. Among the factors that can influence his choices are: the exact nature of the ingredients, active or not, entering in the composition; the condition, the age and the weight of the mammal, the stage of dermatitis or HD and the nature of the treatment.

According to another aspect, the invention proposes the use of a lactic bacterium strain for the manufacture of the composition of the invention. In a preferred embodiment, the Lactobacillus acidophilus strain other than I-1492; and the Lactobacillus casei strain may be of commercial origin and can be purchased from various manufacturers of lactic ferments.

For preparing a composition according to the present invention at least one of the Lactobacillus strains according to the present invention is incorporated in a suitable support, in an amount of from about 10 billions to about 100 billions micro-organisms per unit of about 98 g of the composition.

The composition according to the present invention can be obtained, for example, by fermenting the lactic bacteria in a milk-based medium. For this purpose, a process such as the following may be used. A person skilled in the art will appreciate that other processes may also be suitable.

Firstly, the I-1492, Lactobacillus acidophilus (other than I-1492) and L. casei strains are incubated in a MRS type fermentation medium according to a standard program comprising several steps. The recombined lacteal base is pasteurized for 1.5 minutes at 95° C. and inoculated from 1 to 10% with a mixture of these bacteria. Finally, it is incubated at 37° C. for 7 to 15 hours.

The quantity or the concentration of lactic bacteria which is administered to a human or an animal, or that is present in the composition of the invention is a therapeutically effective quantity. A therapeutically efficient quantity of lactic bacteria is the necessary quantity to obtain positive results without causing excessively negative effects in the host to which the lactic bacteria or the composition is administered. Indeed, an efficient quantity of lactic bacteria to prevent or treat dermatitis or HD is a quantity, which is sufficient to attenuate or reduce in any manner the symptoms linked to dermatitis and/or HD. An effective amount can be administered in one or more administrations (i.e. in a single dose or multiple doses), according to a regimen. For example, as mentioned above, an efficient quantity according to a preferred embodiment of the invention is about 10 to about 100 billions bacteria per unit of about 98 g of the composition. Such a quantity may be administered in a single dose or may be administered by another regime according to which it is efficient. However, it is understood that the exact quantity of lactic bacteria or of each of the components of the composition and the quantity of the composition to be administered will vary according to factors such as the type of dermatitis to prevent or treat, the other ingredients in the composition, the mode of administration, the age and the weight of the mammal, etc.

In one embodiment, the composition is suitable for oral consumption. In this embodiment, the composition is preferably a food product. “Food product” as intended herein will be broadly understood as any component ingestible by an animal such as a human. In this embodiment, the composition according to the present invention can be presented as a solid or a liquid form, usual for pharmaceutical or nutritional administration, i.e. for example liquid forms of administration, in a gel, capsule or any other support known to the person skilled in the art. Among the usable compositions, we can notably cite the compositions that can be administered orally. In the present case, the composition of the invention can be administered as food form (for example a lactic ferment) or as food supplements. More particularly, the composition according to the present invention can be presented in a variety of ingestible forms, such as e.g. a dairy-based product such milk, yogurt, curd, fermented milks and milk based fermented products, soy based fermented products, fermented cereal based product such as a rice-based product, milk based powders and infant formulae. The composition can also be administered in the form of food or food supplements. Such foods may be protein concentrates such as those used in hospitals.

In case of a pharmaceutical preparation the product may be prepared in forms of but not limited to capsules, tablets, liquid bacterial suspensions, dried oral supplements, wet oral supplements, dry tube feeding or a wet tube feeding etc., with the amount of Lactobacillus strains to be incorporated therein being in the range of up to but not limited to 30 billions. In one embodiment, the composition is Bio-K+® capsules.

In one embodiment the composition, whether in the form of a food product, a pharmaceutical composition or any other form, comprises at least 10 billions colony forming unit (CFU) of bacteria, preferably at least 30 billions colony forming unit (CFU) of bacteria, more preferably, at least 50 billions colony forming unit (CFU) of bacteria, and even more preferably at least 100 billions colony forming unit (CFU) of bacteria (e.g. capsule or per 98 grams). Examples of suitable products would include the Bio-K+ regular, strong and extra strength probiotic capsules. The microorganisms in the composition may remain living and active for up to about 2 years under refrigeration.

In one embodiment, the composition administered in the method of the invention is administered in a single dose. According to an alternate embodiment, the composition may be administered in a plurality of doses. Accordingly, the present invention also concerns a method for prevention or treatment of eczema, comprising the step of administering to a mammal an effective amount of composition of the invention. In preferred embodiment the administration is an oral administration. In a preferred embodiment, the composition is administered at the rate of about 49 g per day for the first two days and then at the rate of about 98 g per days for the next period of at least 10 days.

According to a further embodiment, there is provided, together with the method of the invention, the use of moisturizers. When a moisturizer is use it is applied to the external layers of the skin. The moisturizers are compounds or mixture thereof specially designed to make the skin softer and more pliable by increasing its hydration and preventing the skin from becoming too dry or oily. Naturally occurring skin lipids and sterols as well as artificial or natural oils, humectants, emollients, lubricants, etc. may be part of the composition of skin moisturizers. The moisturizer can be applied at least once a day, more preferably it is applied 3 times per day.

According to a further embodiment, there is provided, together with the method of the invention, the use of cleanser. The cleanser is used to remove, dead skin cells, oil, dirt, and other types of pollutants from the skin of the face. This helps to unclog pores and prevent skin conditions that might induce the development of dermatitis. The treatment with the cleanser can be performed at least once a day, more preferably it is applied 3 times per day. When used in combination with a moisturizer, it is preferably the cleanser to be used before the application of the moisturizer.

The present invention also includes useful pharmaceutical kits, for example, for the prevention of dermatitis. The kits comprise one or many containers containing a composition according to the present invention. Such kits may additionally include, if desired, one or many conventional pharmaceutical components like, for example, containers containing one or many pharmaceutically acceptable vehicles, or any other additional containers, which will be evident to a person skilled in the art. A kit according to the present invention can advantageously include instructions in the form of a pamphlet or of any other printed support, indicating the quantities of the compositions to be administered, the instructions for the administration, and/or the instructions to mix the components. Furthermore, the kit of the present invention might include agent like cleansers and/or moisturizers.

The following example serves to illustrate the extent of the use of the present invention and not to limit its scope. Modifications and variations may be made without forgetting the intent and the extent of the invention. Even though other equivalent methods or products to those that are found herein to test or to realize the present invention may be used, the material and the preferred method.

### EXAMPLE 1

### Case Report: Treatment of Hand Dermatitis in Individual

A male patient suffering from hand eczema received Bio-K+® fermented milk probiotic, formulated and produced by Bio-K Plus International Inc, Laval, Québec, Canada. The patient was instructed to store the product at 4° C. The commercially available probiotic, Bio-K+® fermented milk probiotic used in the study, contains 50 billion colony forming units (CFU) of L. acidophilus CL1285® and L. casei LBC80R®.

The Bio-K+® fermented milk probiotic was administered periodically to the patient. Briefly stated, the patient received the Bio-K+® fermented milk probiotic during periods extending over several weeks (the “administration periods”), interrupted by periods of several weeks during which the patient received no probiotics (the “non administration periods”). The administration and non administration periods alternated over several months.

### Results

A direct correlation has been established between the Bio-K+® fermented milk probiotic uptake and the reduction of hand eczema symptoms. The symptoms were maintained constantly low during the periods of administration of the Bio-K+® fermented milk probiotic. However, the HD symptoms increased noticeably during periods the non administration periods, to revert back to low levels when the administration of the Bio-K+® fermented milk probiotic restarted for a new administration period.

### EXAMPLE 2

### Statistical Validation of the Effectiveness of the Composition for the Treatment of Hand Eczema

Thirty patients were enrolled in the 12-week trial at one study center in St. John's, Newfoundland and Labrador, Canada. All research procedures performed in the study were in strict accordance with the study protocol, which had full ethics approval from the central ethic committee of the Research Review Board (Waterloo, Canada).

Male and female patients over 18 years of age with mild to severe HD for 6 months and willing to comply with the relevant treatment and prevention guidelines were included in the study. Female patients of childbearing potential were required to have a negative pregnancy test at baseline and use a reliable contraceptive method throughout the study duration.

Subjects excluded from the study included patients with other concomitant skin conditions of the hands (e.g. irritant or allergic contact dermatitis, dyshidrotic eczema, hyperkeratotic dermatitis) or infected lesions of the hands requiring medical treatment, patients using systemic corticosteroids, non-steroidal anti-inflammatory drugs, immunomodulators, biologics (antibodies, fusion proteins, etc.), oral primrose oil, traditional Chinese herbal therapies, had previously received a heart valve replacement or a serious illness within the 4 weeks preceding the study. Subjects also excluded from the study included those that presented with a medical condition that in the opinion of the investigator would adversely affect their safe participation in the study or affect the conduct of the study, pregnant or nursing females, subject's with a history of substance abuse, and patients with a history of poor compliance with medical treatment.

At screening, the investigator performed a thorough general medical and dermatological examination of the hands and completed a checklist of inclusion and exclusion criteria confirming the eligibility of each subject.

All eligible subjects received consent forms at least 24 hours prior to their first visit. During their baseline visit, subjects were provided with a full study explanation and answers to any questions before consenting to study participation and photographic documentation of their HD.

### Interventions

The study products were made available, formulated and produced by Bio-K Plus International Inc, Laval, Québec, Canada. Patients were instructed to store the product at 4° C. The commercially available probiotic used in the study, were in the form of capsules commercialized under brand name Bio-K+® formula, and contains 30 billion colony forming units (CFU) of L. acidophilus CL1285® and L. casei LBC80R®. Non-medicinal ingredients of the capsules included cellulose, ascorbic acid, magnesium stearate, silicon dioxide and titanium dioxide. Moreover, the capsules were enteric coated. It was administered in a daily dose of one capsule per day.

Subjects were provided with instructions on preventative measures for HD throughout the study's duration and instructed to cleanse and apply a moisturizer at least 3 times a day. This information was repeated at each study visit.

Participants received a two month supply (dispensed in two week allotments at each study visits) of the study product and were instructed to take 1 capsule after breakfast.

### Outcomes

Primary parameters: 1) The time of improvement and time to resolution of HD, and, 2) the extent of disease clearance as assessed by improvements in clinical scores using the Physician Global Assessment (PGA) severity scale, MTLSS and digital photographs as well as patient reported pain; (pain presence (5 point scale); pain frequency (10 point scale Visual Analogue Scale (VAS) comparing day 0 versus week 12 scores.

Secondary parameters: 1) the ease of Bio-K+® use, 2) overall patient well-being and satisfaction during treatment period, and 3) the safety of Bio-K+ therapy, evaluating the treatment related withdrawals and adverse events over the 12 week study period.

### Assessments, PGA and MTLSS Scores

Demographic information for each subject was collected at day 0 (i.e. age, gender, height, weight, etc.) and the HD status. The extent and severity of the patients' HD was assessed at baseline, and at 2, 4, 8 and 12 weeks of treatment. A telephone interview was performed at day 7 to ensure compliance with the study protocol and address any questions.

Localization of HD was checked (yes/no) after which the Physician's Global Assessment (PGA) of overall HD severity was scored for the right and left hand separately and digital photographs were taken. The PGA has been widely used for studies on HD and other types of dermatitis. The PGA scored HD severity in 5 states, from severe to clear (severe; moderate; mild; almost clear; clear), scoring the features of HD (Erythema; scaling; Hyperketatosis/lichenification; Vesiculation; edema; fissures; Puritus/pain) that were present on the palm or dorsum of both hands.

The scoring tool further addressed the intensity of the features present and the size of the area that was affected. Clinical assessment was performed by one of two physicians that conducted the statistical validation study. The PGA includes one symptom (pruritus/pain) that cannot be linked to the photographic guide but is clearly relevant to disease severity and is a major driver for patients to seek medical help. Therefore, MTLSS were used (which looked at the overall diseases in more details on a scoring scale in which 0=none, 1=mild, 2=moderate and 3=severe) these items were scored in more details.

When itchy skin was present this was further specified in: continuously; most of the time; >30% of the day; only after my hand was wet; >10% of the day 8. The presence of pain was reported by the patients using a 5-point scale (1=strongly disagree to 5=strongly agree) and the intensity of the pain was assessed with the 10 point Visual Analogue Scale (VAS) (0 for no pain to 10 for pain being unbearable). For this reason the presence of overall skin dryness, flakiness and pruritus was also scored in more detail (0=none, 1=mild, 2=moderate, 3=severe). In patients reporting the presence of pruritus, they rated the amount of time per day experiencing pruritus and factors that may have influenced it, on a 5-point scale (1=>10% of the day, 2=only after hand is wet, 3=>30% of the day, 4=most of the time, and 5=continuously).

At the end of the 12 week treatment period the outcome of treatment satisfaction of the patients was assessed according to the defined schedule. Patients were asked about their HD, scoring: dry skin (4 point scale); changes to their skin condition during the study period; skin texture; presence of skin irritation; lesions, etc.

Additionally, the patients kept a daily diary of their general well-being, any adverse events and defecation patterns throughout the treatment period.

### Statistical Analyses

The sample size calculated for this study was N=30 and the analyses were performed on the patients results according to the as per protocol principle. Statistical evaluation was performed independently (Technical University, Delft, NL and the Newfoundland and Labrador Centre for Health Information, St. John's, Newfoundland Labrador) using Statistical Package for the Social Sciences (SPSS 18.0). Where appropriate, the paired sample t-test or a one-way repeated measure ANOVA was used. Tests were carried out at the 5% significance level, and a confidence interval of 95%.

The PGA scores were ordinal numeric values ranging from 0-4 and were treated as a continuous variable. The data were analyzed using a one-way ANOVA and was carried out per protocol on a sample of 22 patients. The percentage of change of the PGA severity score between baseline and day 84 was calculated for both left and right hand scores (see Table 1 below). Responses were classified into five categories: no change (0%), slight (1-25%), minimal (26-50%), moderate (51-74%), and significant change (75-100%). Zero or positive values represented ‘no change’, whereas negative values represented an improved response.

TABLE 1
% Change of the PGA Severity Score Comparing Day 0 versus Day 84

| | No (%) patients | |
|---|---|---|
| | Right hand | Left hand |
| No eczema at day 0 | 2* (9%) | 2* (9%) |
| 1 = No change (0%) | 3 (13.6%) | 1 (4.5%) |
| 2 = Slight change (1-25%) | 5 (22.8%) | 6 (27.3%) |
| 3 = Minimal change (26-50%) | 3 (13.6%) | 4 (18.2%) |
| 4 = Moderate change (51-74%) | 4 (18.2%) | 4 (18.2%) |
| 5 = Significant change (75%-100%) | 5 (22.8%) | 5 (22.8%) |

*4 patients presented eczema lesions in a single hand

The MTLSS scores ranged between 0-24 and, thus, were also treated as continuous variable. Early terminated or withdrawn participants were removed from the analysis at the point of termination or withdrawal. Therefore, the sample size used for this analysis has decreased over time of the 84 days trial period. Paired sample t-tests were used during the analysis to evaluate significant changes in patients' response to treatment from baseline to specified time period for each hand. A one-way repeated measures analysis of variance was used to assess overall differences in hand scores over the entire treatment period. Tukey-Kramer post-hoc test were used to compare differences in mean hand scores between specified follow-up periods.

Analyses were completed using both original data and modified data whereby missing values (e.g. early termination) were imputed by carrying forward last recorded treatment score. P-values<0.05 were considered significant.

### Withdrawal Rates

The number of subjects withdrawn was listed in full stating the reason for withdrawal. The number of adverse events was listed in full stating the classification of the adverse event and if there was a relation to the treatment. All the patients that had withdrawn consent were considered having a negative outcome of the study.

### Results

### Patient's Characteristics at Baseline

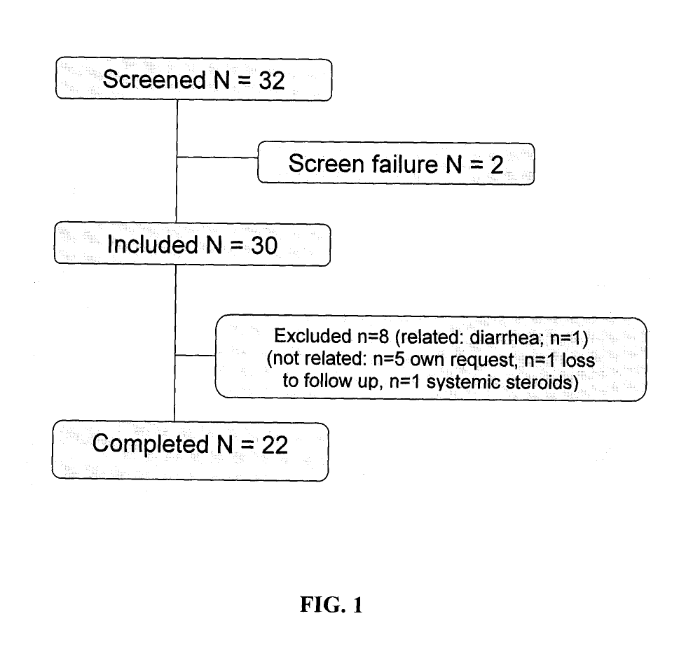

A total of N=32 were screened from the study center from September 2009 till November 2010, as seen in FIG. 1. Two subjects were not included due to a screen failure. Thirty two patients consented (of which 2 screened failed) and of which N=22 completed the study period of 12 weeks. FIG. 1 shows the subject consort flow diagram. The majority, of the participants that finished the study were female (n=15 (68%), the mean age was 48.82 (SD±14.68; range 24-78) years. Subjects had their first outbreak of eczema at a mean age of 35.9 (SD±16.22; range 21-65) years. At day 0, most patients had HD on both hands, however two patients had only their right or their left hand affected.

All patients were in reasonably good health with 7 participants reporting a tobacco smoking habit at baseline (mean of 5 packs/week). At the start of the study, 12 participants (40%) reported regularly using a moisturizer for HD control (see Table 2 below).

TABLE 2
Demographics and Patient' Characteristics

| | | Consenting participants | |
|---|---|---|---|
| | | (N = 32/30) | |
| Socio-demographic Data−+ | | (2 screened failed) | |
| Age (±SD) | | 48.82 ± 14.68 | |
| Gender | Male (%) | 7 | (32%) |
| | Female (%) | 15 | (68%) |

| | | |
|---|---|---|
| First Outbreak (age) | 35.88 | (+16.22) |
| Participants reporting a tobacco | 7 | (mean: 5 packs/week) |
| smoking habit at baseline | | |
| Participants reporting regularly | 12 | (40%) |
| using a moisturizer for HD | | |
| control | | |

### Withdrawal Rates

The only adverse event was a case of mild diarrhea. The other 7 patients that failed to complete the study did so for various reasons (5 withdrew consent, 1 was lost to follow-up, 1 patient required systemic corticosteroids). Three of the 5 patients that withdrew consent were in the study for at least 8 weeks (FIG. 1).

### Primary Outcomes

The dermatitis lesions had already improved significantly at day 14 in the right hands (mean score at day 0: 9.09 (±5.68) versus 7.09 (±4.51) at day 14 (p<0.01) and the left hands (mean score at day 0: 8.91 (±5.90) versus 6.77 (±4.25) (p<0.003). These results are shown in Table 3 below. The improvement of the dermatitis persisted in both hands until day 84 visit as demonstrated by the significant decrease of the PGA scores in Table 3.

TABLE 3
Evolution of Skin Condition Day 0 (Start) versus Day 84 (End)

| | | | | | | P Value |
|---|---|---|---|---|---|---|
| Location | Day 0: | Day 14: | Day 28: | Day 56: | Day 84: | Day 0 |
| and PGA | Mean | Mean | Mean | Mean | Mean | vs |
| Score | (±SD) | (±SD) | (±SD) | (±SD) | (±SD) | Day 84 |
| Right | 9.09 (5.68) | 7.09 (4.51) | 6.45 (4.84) | 6.09 (5.11) | 7.36 (5.30) | P < 0.04 |
| hand | | P < 0.01 | | | | |
| Left hand | 8.91 (5.90) | 6.77 (4.25) | 5.77 (3.82) | 6.14 (5.37) | 6.63 (5.29) | P < 0.01 |
| | | P < 0.003 | | | | |

Table 3 indicates that a more than 50% improvement was present in either hand of more than 40% of patients between day 0 and 84 when no change was noticed in a much smaller number of patients. The PGA global score for both hands was decreased in 54.5% of patients including 22.7% of them being cleared of any lesion (Data Not Shown).

The MTLSS scores for the left hand were significantly decreased from 9.19 to 7.31 at day 14 (see Table 4A) and the same was present in the right hand, the decrease being from 9.15 to 7.59 (see Table 4B). These decreases persisted until the end of the study, and remained statistically significant until day 56.

TABLE 4A
(MTLSS) Patient's Left Hand Response to Probiotic (Treatment from
baseline to Specified Time Period)

| | | | | | 95% Confidence | | |
|---|---|---|---|---|---|---|---|
| | | | Sim- | Mean | Interval of the | | |
| Left | | | ple | Paired | difference | | P |
| Hand | | Mean | Size | Differences | Lower | Upper | Value |
| Pair 1 | Baseline | 9.19 | 26 | 1.88 | .407 | 3.362 | 0.015 |
| | Day 14 | 7.31 | 26 | | | | |
| Pair 2 | Baseline | 9.54 | 24 | 2.79 | .635 | 4.948 | 0.013 |
| | Day 28 | 6.75 | 24 | | | | |
| Pair 3 | Baseline | 9.33 | 24 | 2.75 | .027 | 5.473 | 0.048 |
| | Day 56 | 6.58 | 24 | | | | |
| Pair 4 | Baseline | 9.33 | 21 | 2.57 | −.518 | 5.661 | 0.098 |
| | Day 84 | 6.76 | 21 | | | | |

TABLE 4B
(MTLSS) Patient's Right Hand Response to Probiotic (Treatment from
baseline to Specified Time Period)

| | | | | | 95% Confidence | | |
|---|---|---|---|---|---|---|---|
| | | | Sim- | Mean | Interval of the | | |
| Right | | | ple | Paired | difference | | P |
| Hand | | Mean | Size | Differences | Lower | Upper | Value |
| Pair 1 | Baseline | 9.15 | 27 | 1.56 | .136 | 2.975 | 0.033 |
| | Day 14 | 7.59 | 27 | | | | |
| Pair 2 | Baseline | 9.19 | 26 | 2.46 | .865 | 4.058 | 0.004 |
| | Day 28 | 6.73 | 26 | | | | |
| Pair 3 | Baseline | 9.32 | 25 | 2.80 | .596 | 0.596 | 0.015 |
| | Day 56 | 6.52 | 25 | | | | |
| Pair 4 | Baseline | 9.09 | 22 | 1.73 | −.612 | 4.066 | 0.140 |
| | Day 84 | 7.36 | 22 | | | | |

### Secondary Outcome Measures:

Only one subject was withdrawn due to a study related, event (diarrhea), which was not severe, indicating Bio-K+® as treatment of patients with HD to be safe. After discontinuing Bio-K+® the diarrhea had subsided within two days. Patients reported that taking the dose of Bio-K+® once per day (at breakfast) to be easy and reported no discomfort from using the product (see FIG. 1).

Although the above description relates to a specific embodiment as presently contemplated by the inventor, it will be understood that the invention described herein in its broad aspect includes mechanical and functional equivalents of the elements described herein.

1. A method of treating or preventing eczema in a human in need thereof, the method comprising administering to said human a composition comprising a mixture of Lactobacilli, wherein the mixture comprises at least one strain of Lactobacillus acidophilus and at least one strain of Lactobacillus casei strain.

2. The method according to claim 1, wherein said at least one strain of Lactobacillus acidophilus comprises Lactobacillus acidophilus strain I-1492 deposited at the CNCM.

3-4. (canceled)

5. The method according to claim 1, wherein said composition further comprises a biologically acceptable carrier.

6. The method according to claim 5, wherein said biologically acceptable carrier is selected from the group consisting of pharmaceutically acceptable carriers and nutritionally acceptable carriers.

7-8. (canceled)

9. The method of claim 1, wherein said composition is administered orally.

10. The method according to claim 9, wherein said composition is formulated as a food product.

11. The method according to claim 10, wherein said food product is selected from the group consisting of lyophilized powders, dairy-based products, soy-based products and rice-based products.

12. The method of claim 11, wherein said food product is a lactic ferment product.

13. The method according to claim 9, wherein said composition is administered in a capsule.

14. The method according to claim 1, wherein said composition comprises between 10 to 100 billions colony forming unit (CFU) of Lactobacilli.

15-19. (canceled)

20. The method according to claim 14, wherein said composition is administered once a day.

21. (canceled)

22. The method according to claim 1, wherein said eczema is hand eczema.

23. The method according to claim 1, wherein said eczema is a mild eczema.

24. The method according to claim 1, wherein said eczema is a moderate eczema.

25. The method according to claim 1, wherein said eczema is a severe eczema.

26-53. (canceled)

54. The method of according to claim 1, comprising administering said composition at least once a day for at least two weeks.

55. The method of according to claim 1, further comprising applying a moisturizer, and/or a cleanser or combination thereof to the skin of said human.

56. A method of treating or preventing eczema in a human in need thereof, the method comprising administering orally to said human a lactic ferment comprising Lactobacillus acidophilus strain I-1492 deposited at the CNCM and at least one strain of the Lactobacillus casei group.

57. The method of according to claim 56, comprising administering at least 30 billion colony forming unit (CFU) of Lactobacilli at least once a day for at least two weeks.

58. A method of treating or preventing eczema in a human in need thereof, the method comprising administering to said human at least one strain of Lactobacillus acidophilus and at least one strain of Lactobacillus casei.
